(19) United States
(12) Patent Application Publication
(10) Pub. No.: US 2025/0082861 A1
(43) Pub. Date: Mar. 13, 2025

(54) SAFE INJECTION SYSTEMS AND METHODS

(71) Applicant: CREDENCE MEDSYSTEMS, INC., Menlo Park, CA (US)

(72) Inventors: Alan E. Shluzas, San Carlos, CA (US); Everett Matthew Notaro, Santa Clara, CA (US)

(73) Assignee: CREDENCE MEDSYSTEMS, INC., Menlo Park, CA (US)

(21) Appl. No.: 18/830,546

(22) Filed: Sep. 10, 2024

Related U.S. Application Data
(60) Provisional application No. 63/537,641, filed on Sep. 11, 2023.

(51) Int. Cl. A61M 5/32

(52) U.S. Cl. CPC A61M 5/322; A61M 5/3293

(57) ABSTRACT

A system for injection includes a syringe body having proximal and distal ends and defining a syringe interior, a stopper member disposed in the syringe interior, a needle hub assembly coupled to the syringe body at the distal end thereof, and a plunger member coupled to the stopper member and configured to be manipulated to insert the stopper member distally in the syringe interior relative to the syringe body and the needle hub. The plunger member includes a plunger body defining a plunger interior, a retraction member disposed in the plunger interior, and a spring disposed in the plunger interior. The retraction member includes a catch-latch and a trigger movably disposed in the catch-latch along a longitudinal axis thereof. The trigger defines a proximal radial extension, a distal radial extension, and an annular trough between the proximal and distal radial extensions.

The present application claims priority to U.S. Provisional Patent Application Ser. No. 63/537,641, filed on Sep. 11, 2023 under attorney docket number CM.30037.01 and entitled “SAFE INJECTION SYSTEMS AND METHODS.” This application includes subject matter similar to the subject matter described in the following co-owned U.S. patent applications: (1) U.S. patent application Ser. No. 14/321,706, filed Jul. 1, 2014 and issued as U.S. Pat. No. 9,814,842 on Nov. 14, 2017 under attorney docket number CM.20001.00 and entitled “SAFETY SYRINGE”; (2) U.S. patent application Ser. No. 14/543,787, filed Nov. 17, 2014 and issued as U.S. Pat. No. 10,300,217 on May 28, 2019 under attorney docket number CM.20002.00 and entitled “SYSTEM AND METHOD FOR DRUG DELIVERY WITH A SAFETY SYRINGE”; (3) U.S. patent application Ser. No. 14/696,342, filed Apr. 24, 2015, and issued as U.S. Pat. No. 10,010,677 on Jul. 7, 2018 under attorney docket number CM.20003.00 and entitled “SYSTEM AND METHOD FOR SAFETY SYRINGE”; (4) U.S. patent application Ser. No. 15/801,239, filed on Nov. 1, 2017 and issued as U.S. Pat. No. 10,926,038 on Feb. 23, 2021 under attorney docket number CM.20011.00 and entitled “SYSTEM AND METHOD FOR SAFETY SYRINGE”; (5) U.S. patent application Ser. No. 15/801,259, filed on Nov. 1, 2017, and issued as U.S. Pat. No. 10,864,330 on Dec. 15, 2020 under attorney docket number CM.20012.00 and entitled “SYSTEM AND METHOD FOR SAFETY SYRINGE”; (6) U.S. patent application Ser. No. 15/801,281 filed on Nov. 1, 2017 and issued as U.S. Pat. No. 10,912,894 on Feb. 9, 2021 under attorney docket number CM.20013.00 and entitled “CARTRIDGE SAFETY INJECTION SYSTEM AND METHODS”; (7) U.S. patent application Ser. No. 15/801,304 filed on Nov. 1, 2017 and issued as U.S. Pat. No. 10,960,144 on Mar. 30, 2021 under attorney docket number CM.20015.00 and entitled “SYSTEM AND METHOD FOR SAFETY SYRINGE”; (8) U.S. patent application Ser. No. 16/435,429 filed on Jun. 7, 2019 under attorney docket number CM.20019.00 and entitled “SYSTEM AND METHOD FOR SAFETY SYRINGE”; (9) U.S. patent application Ser. No. 16/798,188, filed on Feb. 21, 2020 under attorney docket number CM.20023.00 and entitled “SYSTEM AND METHOD FOR SAFETY SYRINGE”; (10) U.S. patent application Ser. No. 16/837,835, filed Apr. 1, 2020 under attorney docket number CM.20025.00 and entitled “POLYMERIC INJECTION SYSTEMS”; (11) U.S. patent application Ser. No. 16/908,531 filed on Jun. 22, 2020 under attorney docket number CM.20026.00 and entitled “INJECTION SYSTEM AND METHOD”; (12) U.S. patent application Ser. No. 17/031,108 filed on Sep. 24, 2020 under attorney docket number CM.20027.00 and entitled “SYSTEM AND METHOD FOR SAFETY SYRINGE”; (13) U.S. patent application Ser. No. 17/364,546 filed on Jun. 30, 2021 under attorney docket number CM.20028.00 and entitled “SYSTEM AND METHOD FOR SAFETY SYRINGE”; (14) U.S. Provisional Patent Application Ser. No. 63/094,313 filed on Oct. 20, 2020 under attorney docket number CM.30030.00 and entitled “RETRACTION MECHANISM FOR SAFE INJECTION SYSTEM”; (15) U.S. Provisional Patent Application Ser. No. 63/300,394, filed on Jan. 18, 2022 under attorney docket number CM.30034.00 and entitled “INJECTION SYSTEM AND METHOD”. The contents of the applications and patents identified herein are fully incorporated herein by reference as though set forth in full.

### FIELD OF THE DISCLOSURE

The present disclosure relates generally to injection systems, devices, and processes for facilitating various levels of control over injection processes, and more particularly to devices and methods related to safe injection systems, devices, and processes that retract a needle at least partially within a plunger member after injection.

### BACKGROUND

Millions of syringes, such as that depicted in FIG. 1A (2), are consumed in healthcare environments every day. A typical syringe (2) comprises a tubular body (4), a plunger (6), and an injection needle (8). As shown in FIG. 1B, such a syringe (2) may be utilized not only to inject fluid into a patient, but also to withdraw or expel fluid out of or into a container such as a medicine bottle, vial, bag, or other drug containment system (10). Indeed, due to regulatory constraints in some countries such as the United States as well as sterility maintenance concerns, upon use of a medicine bottle (10) with a syringe (2) as shown in a particular patient's environment, such medicine bottle may only be utilized with a single patient and then must be disposed of—causing significant medical waste from bottle and remaining medicine disposal, and even contributing to periodic shortages of certain critical drugs. Referring to FIG. 2A, three Luer-type syringes (12) are depicted, each having a Luer fitting geometry (14) disposed distally, so that they may be coupled with other devices having similar mating geometry, such as the Luer manifold assembly (16) depicted in FIG. 2B. The Luer manifold assembly of FIG. 2B may be used to administer liquid drugs to the patient intravenously with or without the use of an intravenous infusion bag. The Luer fittings (14) of the syringes of FIG. 2A may be termed the “male” Luer fittings, while those of FIG. 2B (18) may be termed the “female” Luer fittings; one of the Luer interfaces may be threaded (in which case the configuration may be referred to as a “Luer lock” configuration) so that the two sides may be coupled by relative rotation, which may be combined with compressive loading. In other words, in one Luer lock embodiment, rotation, possibly along with compression, may be utilized to engage threads within the male fitting (14) which are configured to engage a flange on the female fitting (18) and bring the devices together into a fluid-sealed coupling. In another embodiment, tapered interfacing geometries may be utilized to provide for a Luer engagement using compression without threads or rotation (such a configuration may be referred to as a “slip-on” or “conical” Luer configuration). While such Luer couplings are perceived to be relatively safe for operators, there is risk of medicine spilling/leaking and parts breakage during assembly of a Luer coupling. The use of needle injection configurations, on the other hand, carries with it the risk of a sharp needle contacting or stabbing a person or structure that is not desired. For this reason, so called “safety syringes” have been developed.

One embodiment of a safety syringe (20) is shown in FIG. 3, wherein a tubular shield member (22) is spring biased to cover the needle (8) when released from a proximal/retracted position relative to the syringe body (4). The tubular needle shield (22) is “locked” in the distal/extended configuration, such that the needle shield (22) can no longer be returned to the proximal/retracted position, to prevent accidental needle sticks after injection.

Another embodiment of a safety syringe (24) is shown in FIGS. 4A-4B. With such a configuration, after full insertion of the plunger (6) relative to the syringe body (4), the retractable needle (26) is configured to retract (28, 26) back to a safe position within the tubular body (4), as shown in FIG. 4B. Such a configuration which is configured to collapse upon itself may be associated with blood spatter/aerosolization problems, the safe storage of pre-loaded energy which may possibly malfunction and activate before desirable, loss of accuracy in giving full-dose injections due to residual dead space within the spring compression volume, and/or loss of retraction velocity control which may be associated with pain and patient anxiety.

Further complicating the syringe marketplace is an increasing demand for prefilled syringe assemblies such as those depicted in FIGS. 5A and 5B, which generally comprise a syringe body, or “drug enclosure containment delivery system”, (34), a plunger tip, plug, or stopper (36), and a distal seal or cap (35) which may be fitted over a Luer type interface (FIG. 5A shows the cap 35 in place; FIG. 5B has the cap removed to illustrate the Luer interface 14). Liquid medicine may reside in the volume, or medicine reservoir, (40) between the distal seal and the distal end (37) of the plunger tip (36). The plunger tip (36) may comprise a standard butyl rubber material and may be coated, such as with a biocompatible lubricious coating (e.g., polytetrafluoroethylene (“PTFE”)), to facilitate preferred sealing and relative motion characteristics against the associated syringe body structure and material. The proximal end of the syringe body (34) in FIG. 5B comprises a conventional integral syringe flange (38), which is formed integral to the material of the syringe body (34). The flange (38) is configured to extend radially from the syringe body (34) and may be configured to be a full circumference, or a partial circumference around the syringe body (34). A partial flange is known as a “clipped flange” while the other is known as a “full flange.” The flange is used to grasp the syringe with the fingers to provide support for pushing on the plunger to give the injection. The syringe body (34) preferably comprises a translucent material such as a glass or polymer. To form a contained volume within the chamber or reservoir (40), and to assist with expulsion of the associated fluid through the needle, a plunger tip (36) may be positioned within the syringe body (34). The syringe body (34) may define a substantially cylindrical shape (i.e., so that a plunger tip 36 having a circular cross-sectional shape may establish a seal against the syringe body (34)), or be configured to have other cross-sectional shapes, such as an ellipse.

Such assemblies are desirable because they may be standardized and produced with precision in volume by the few manufacturers in the world who can afford to meet all of the continually changing regulations of the world for filling, packaging, and medicine/drug interfacing materials selection and component use. Such simple configurations, however, generally will not meet the new world standards for single-use, safety, auto-disabling, and anti-needle-stick. Thus, certain suppliers have moved to more “vertical” solutions, such as that (41) featured in FIG. 5C, which attempts to meet all of the standards, or at least a portion thereof, with one solution; as a result of trying to meet these standards for many different scenarios, such products may have significant limitations (including some of those described above in reference to FIGS. 3-4B) and relatively high inventory and utilization expenses.

Some safe injection systems such as those described in U.S. Pat. No. 10,010,677, which was previously incorporated by reference herein, include a spring to generate force to retract a needle and a spring latch to hold the spring in a compressed state until an appropriate time to retract the needle after injection. Some spring latches unevenly distribute the force from the spring to the plunger member, thereby causing the plunger member to warp over time, which would impede the function of the safe injection systems. Accordingly, there exists a need for safe injection systems with spring latches that address these limitations.

### SUMMARY

Embodiments are directed to injection systems. In particular, the embodiments are directed to safe injection systems.

In one embodiment, a system for injection includes a syringe body having proximal and distal ends and defining a syringe interior. The system also includes a stopper member disposed in the syringe interior. The system further includes a needle hub assembly coupled to the syringe body at the distal end thereof. The needle hub assembly includes a needle hub, and a needle coupled to the needle hub and having a needle proximal end. Moreover, the system includes a plunger member coupled to the stopper member and configured to be manipulated to insert the stopper member distally in the syringe interior relative to the syringe body and the needle hub. The plunger member includes a plunger body defining a plunger interior, a retraction member disposed in the plunger interior and configured to couple to the needle proximal end, and a spring disposed in the plunger interior and configured to withdraw the retraction member proximally in the plunger interior, the spring having a compressed state and a released state. The retraction member includes a catch-latch and a trigger movably disposed in the catch-latch along a longitudinal axis thereof. The catch-latch has a plurality of distal arms configured to interfere with the needle proximal end, and a plurality of spring latches configured to hold the spring in the compressed state. The trigger defines a proximal radial extension, a distal radial extension, and an annular trough between the proximal and distal radial extensions.

In one or more embodiments, the plurality of spring latches includes first and second spring latches disposed on radially opposite sides of the catch-latch. The first and second spring latches may equally distribute a force from the spring, through the catch-latch, to the plunger body.

In one or more embodiments, the plunger body defines a plurality of openings corresponding to the plurality of spring latches and a plurality of distally facing surfaces defining respective proximal ends of respective ones of the plurality of openings. Each spring latch of the plurality of spring latches may include a proximally facing ramp configured to cooperate with a corresponding distally facing surface of a corresponding opening under a proximally directed force from the spring on the catch-latch to push the spring latch radially inward. Each spring latch of the plurality of spring latches may include a notch configured to facilitate rotation of the spring latch toward and away from the trigger.

In one or more embodiments, the catch-latch includes first and second longitudinal pillars disposed on radially opposite sides of the catch-latch, and a ring coupled to respective proximal ends of the first and second longitudinal pillars and disposed proximal of the plurality of spring latches. The ring may be configured to tilt orthogonal to the first and second pillars to align the retraction member with the plunger body. The catch-latch may define a plurality of windows therein.

In one or more embodiments, the trigger includes a radially extending ring, and the catch-latch includes a plurality of radially inwardly extending bumps configured to interfere with the radially extending ring to temporarily resist axial movement of the trigger relative to the catch-latch. The plurality of radially inwardly extending bumps may include first, second, and third radially inwardly extending bumps. The second radially inwardly extending bump may be disposed on an opposite side of the catch-latch from the first and third radially inwardly extending bumps. The radially extending ring and the plurality of radially inwardly extending bumps may be configured to provide a plurality of rest configurations during assembly of the retraction member. The trigger may include a second radially extending ring. The second radially extending ring and the plurality of radially inwardly extending bumps may be configured to prevent premature conversion of the spring from the compressed state to the released state during an accidental drop of the system.

In one or more embodiments, the needle proximal end defines a radially expanded member at a proximal end thereof, and the catch-latch defines a plurality of rotatable needle latch arms configured to interfere with the radially expanded member to allow the radially expanded member to move proximally past the plurality of rotatable needle latch arms while preventing the radially expanded member from moving distally past the plurality of rotatable needle latch arms. The catch-latch may also define a distal block configured to interfere with the plurality of rotatable needle latch arms to prevent the plurality of rotatable needle latch arms from rotating distally past the distal block. The distal block may define an opening therethrough configured to receive the needle. The needle may also have a tubular joining member configured to receive a distal end of the needle proximal end, and wherein the needle proximal end defines one or more channels at the distal end thereof.

The aforementioned and other embodiments of the invention are described in the Detailed Description which follows.

### BRIEF DESCRIPTION OF THE DRAWINGS

This patent or application file contains at least one drawing executed in color. Copies of this patent or patent application publication with color drawings will be provided by the U.S. Patent and Trademark Office upon request and payment of the necessary fee.

FIGS. 1A to 5C illustrate various aspects of conventional injection syringe configurations.

FIG. 6 is a perspective view of a safe injection system according to some embodiments.

FIG. 7 is an exploded view of a needle hub assembly before it is assembled onto a syringe body according to some embodiments.

FIG. 8 is an exploded view of a needle hub assembly according to some embodiments.

FIG. 9 is an exploded view of a needle assembly according to some embodiments.

FIG. 10 is a perspective view of a needle hub assembly mounted onto a syringe body of a safe injection system according to some embodiments.

FIG. 11 is an exploded view of a retraction member and a plunger member according to some embodiments.

FIG. 12 are perspective views of a catch-latch (top) and a trigger (bottom) according to some embodiments.

FIG. 13 are perspective views of a trigger assembled into a catch-latch in latched (top) and unlatched (bottom) states according to some embodiments.

FIG. 14 are side views of a catch-latch in latched (top) and unlatched (bottom) states according to some embodiments.

FIGS. 15 to 21 are longitudinal cross-sectional views depicting a method of using a safe injection system to perform an injection and to retract a needle after the injection for safe disposal of the used system according to some embodiments.

FIGS. 22 and 23 are perspective views triggers according to some embodiments.

FIG. 24 are side views of a catch-latch in latched (top) and unlatched (bottom) states according to some embodiments.

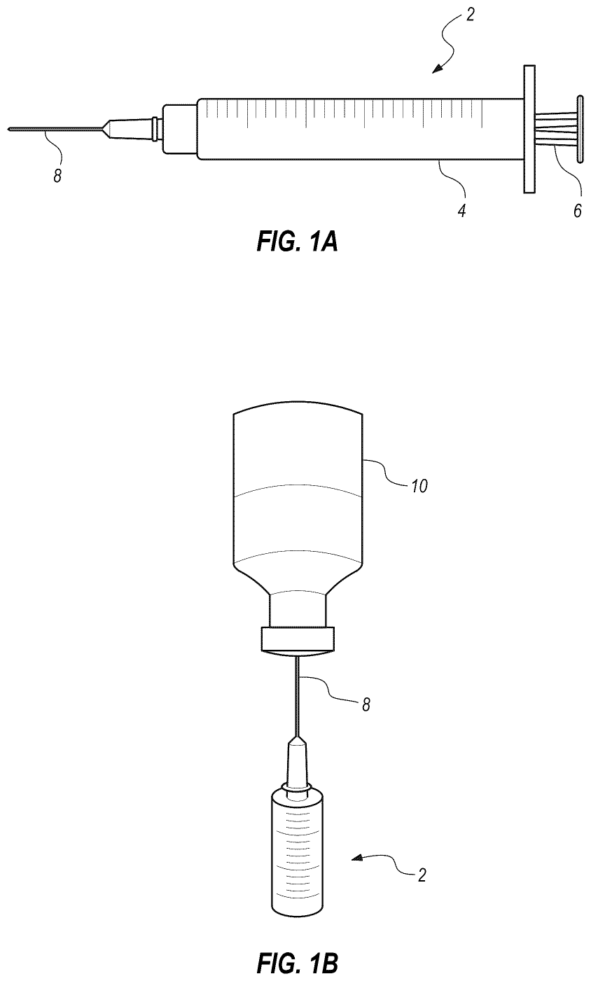

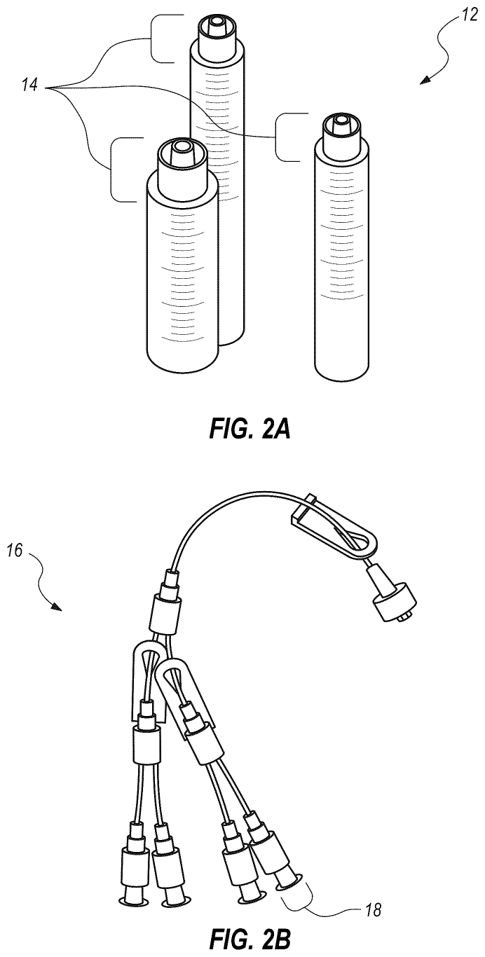

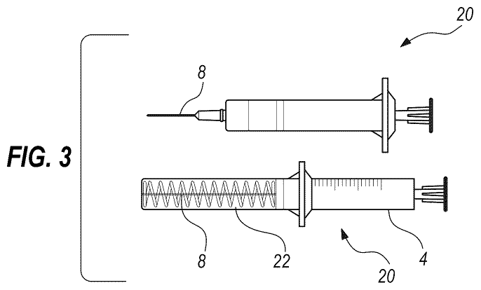

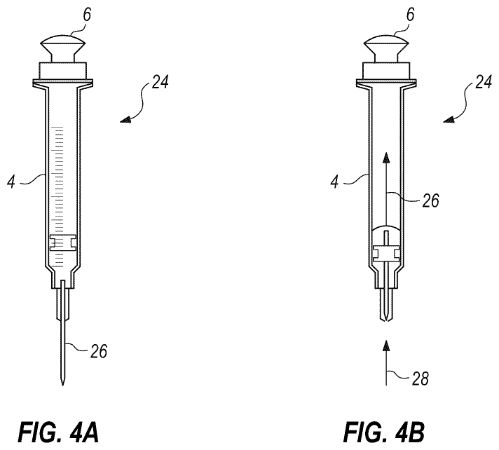

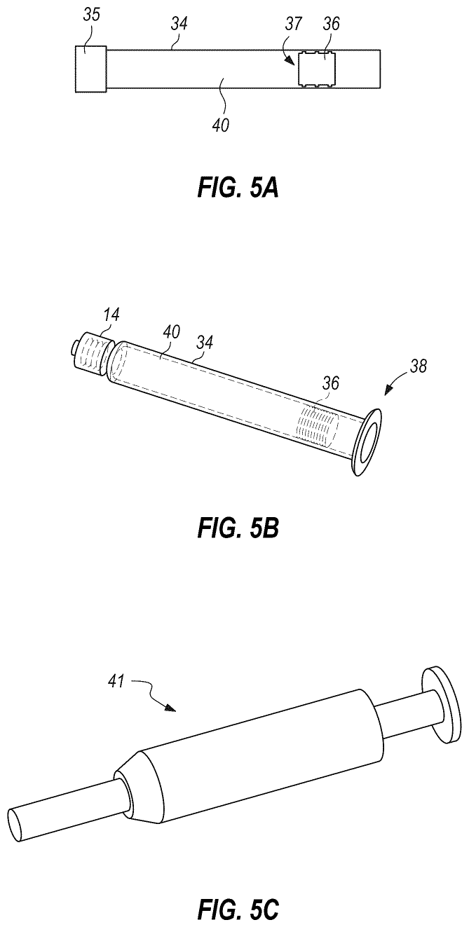

In order to better appreciate how to obtain the above-recited and other advantages and objects of various embodiments, a more detailed description of embodiments is provided with reference to the accompanying drawings. It should be noted that the drawings are not drawn to scale and that elements of similar structures or functions are represented by like reference numerals throughout. It will be understood that these drawings depict only certain illustrated embodiments and are not therefore to be considered limiting of scope of embodiments.

### DETAILED DESCRIPTION OF ILLUSTRATED EMBODIMENTS

### Exemplary Safe Injection System

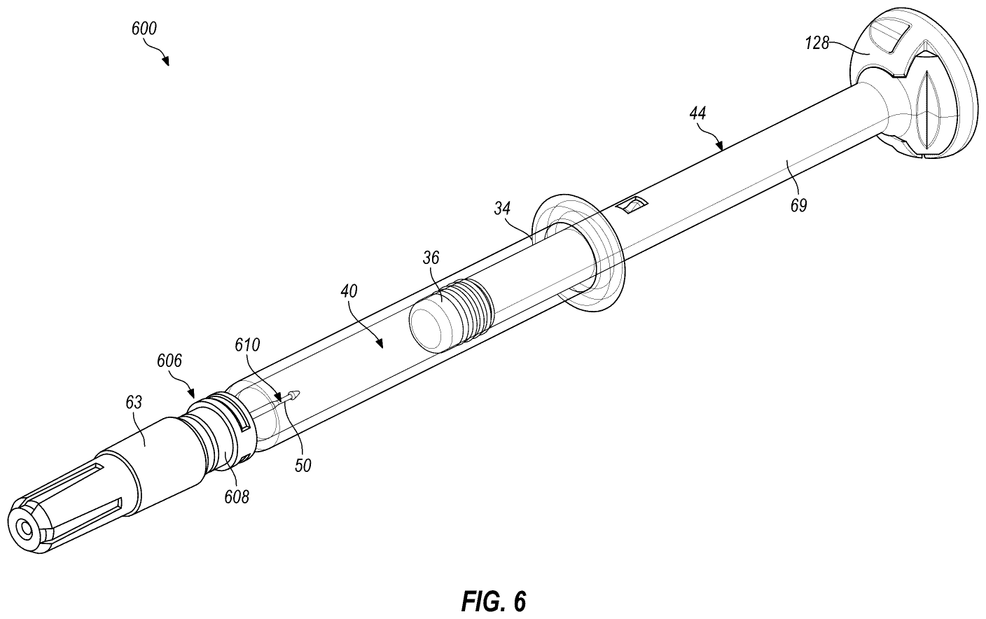

Referring to FIG. 6, a perspective view of a safe injection system (600) is shown, with a conventional off-the-shelf pre-filled syringe body (34) with a conventional stopper member (36) disposed therein according to some embodiments. The stopper member (36) together with the syringe body (34) define a syringe chamber (40). The stopper member (36) occludes a proximal end of the syringe chamber (40). The safe injection system (600) also includes a plunger member (44) coupled to a proximal and of the stopper member (36), for instance with a conventional screw-on connection. A needle hub assembly (606) is disposed at the distal end of the syringe chamber (40) with a needle cover (63) installed for storage.

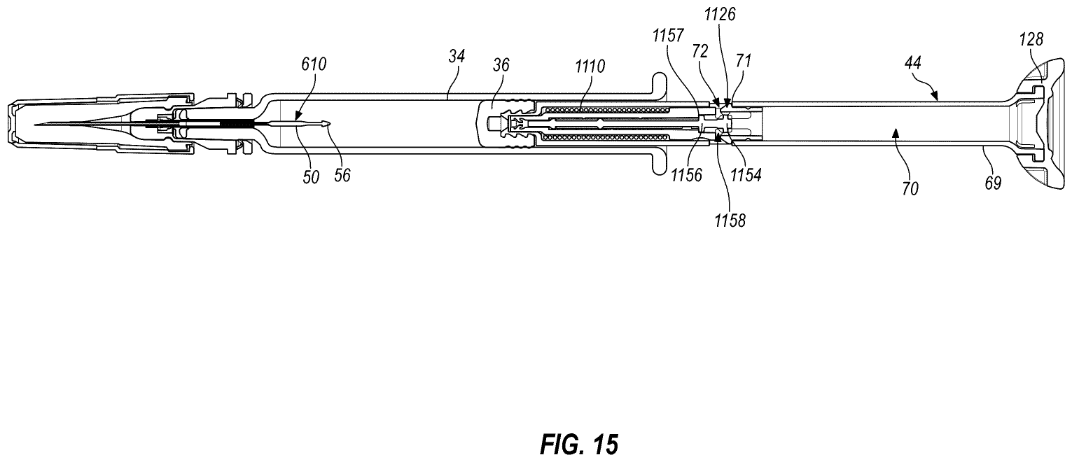

The safe injection system (600) controls injection of a fluid from the syringe chamber (40) through the needle hub assembly (606) driven by distal insertion of the plunger member (44) relative to the syringe body (34) by a user. The plunger member (44) includes a plunger housing member (69) defining a plunger interior (70; see FIG. 15) and a plunger manipulation interface (128) coupled to the plunger housing member (69) at a proximal end thereof.

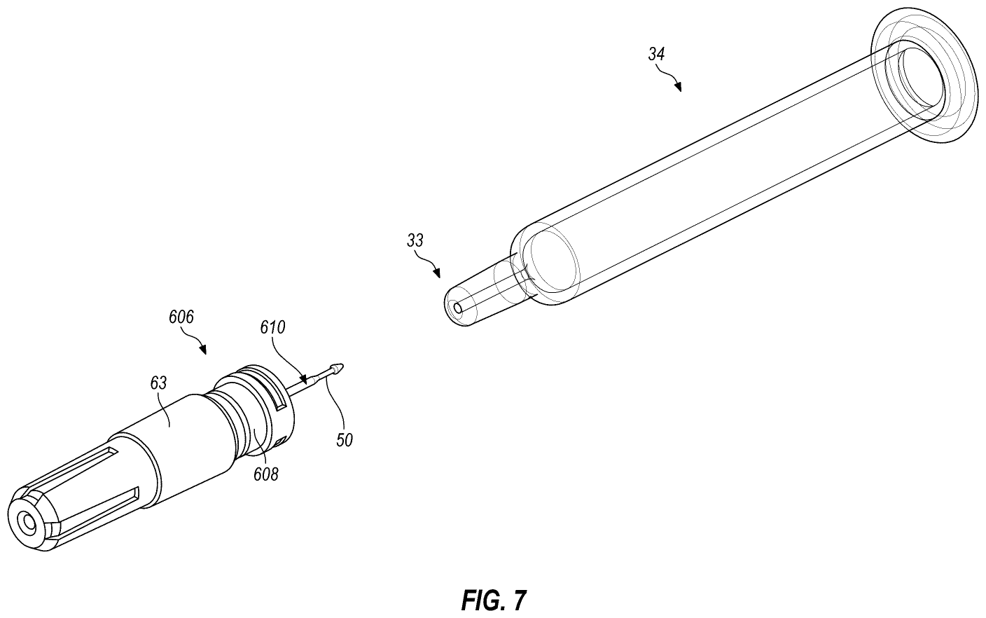

The safe injection system (600) has a staked needle configuration wherein upon presentation to the user, a needle hub assembly (606), comprising a needle hub (608) and a needle assembly (610), is mounted in position ready for injection after removal of a needle cover (63). FIG. 7 is an exploded view of the needle hub assembly (606) before it is mounted onto the syringe body (34) according to some embodiments. The distal tip of the syringe body (34) comprises a luer taper interface (33)

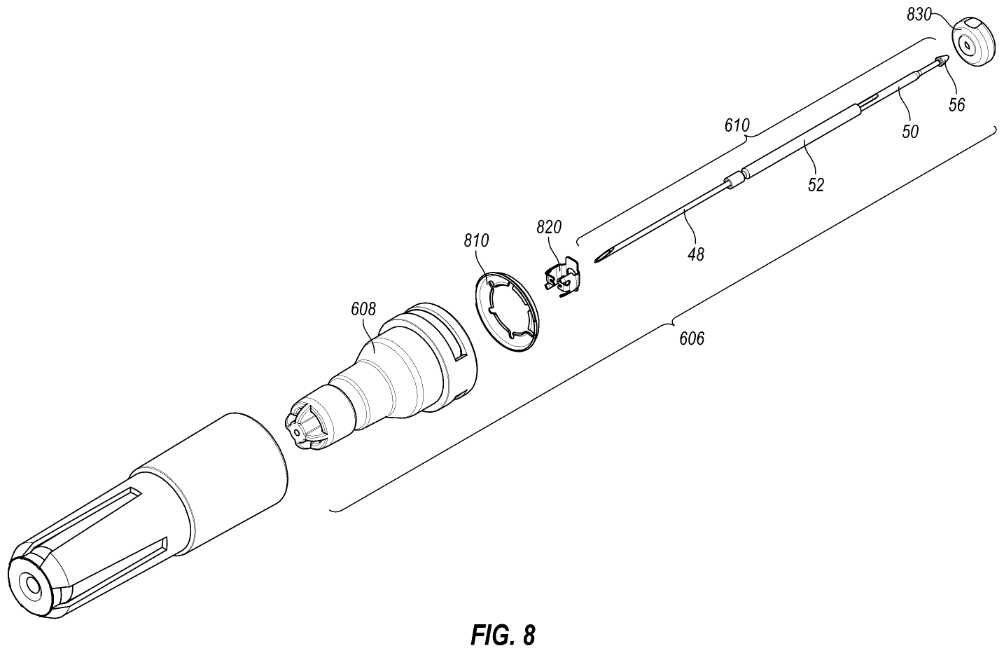

FIG. 8 is an exploded view of the needle hub assembly (606) according to some embodiments. The needle hub (608) includes a lock ring (810) configured to secure the needle hub (608) onto a distal end of the syringe body (34) (see FIG. 6). The needle hub (608) also includes a needle latch member (820) configured to removably couple the needle assembly (610) to the needle hub (608). The needle hub (608) further includes a gasket (830) configured to minimize fluid leakage around the outside of the needle assembly (610). The gasket is positioned between the tip of the luer interface and the inside of the needle hub, around the needle assembly (610) and configured to prevent the contents of the drug chamber from leaking out during storage of the syringe and during injection of the medicine into the patient.

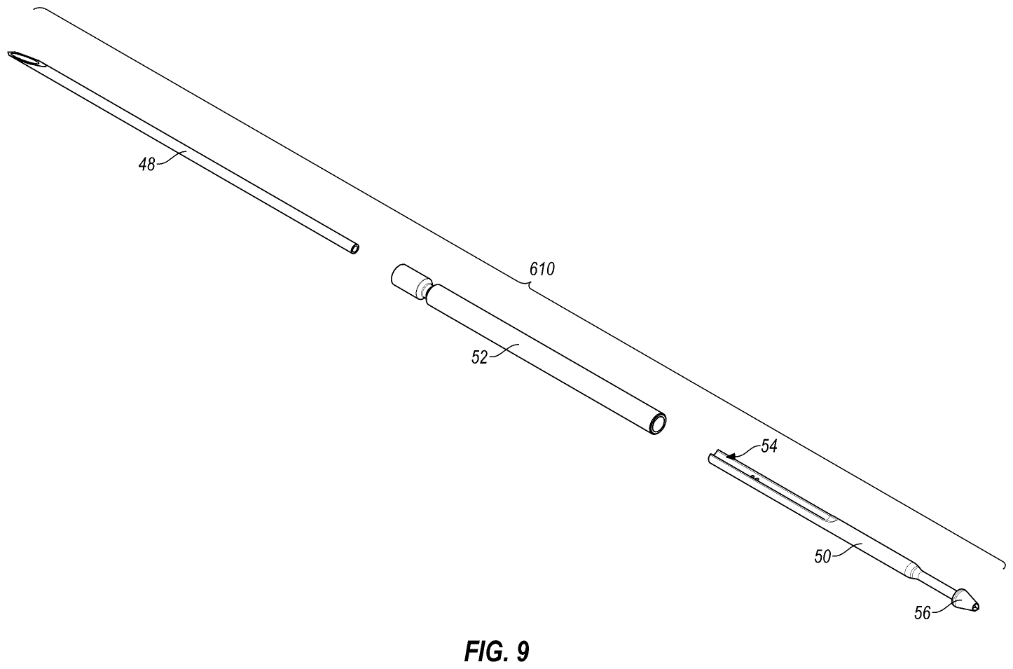

FIG. 9 is an exploded view of the needle assembly (610) according to some embodiments. The needle assembly (610) includes a needle distal end (48) and a needle proximal end (50) coupled to each other by a needle joining member (52). A distal end of the needle proximal end (50) defines a pair of longitudinal channels (54), which form a fluid path from the syringe chamber (40; see FIG. 6), through the longitudinal channels (54), through the needle joining member (52), through the needle distal end (48), and to an exterior of the safe injection system (600, see FIG. 6). While a pair of longitudinal channels is shown, an alternative embodiment may be one longitudinal channel formed in the needle proximal end (50). Additionally, three or more longitudinal channels may be formed in the needle proximal end.

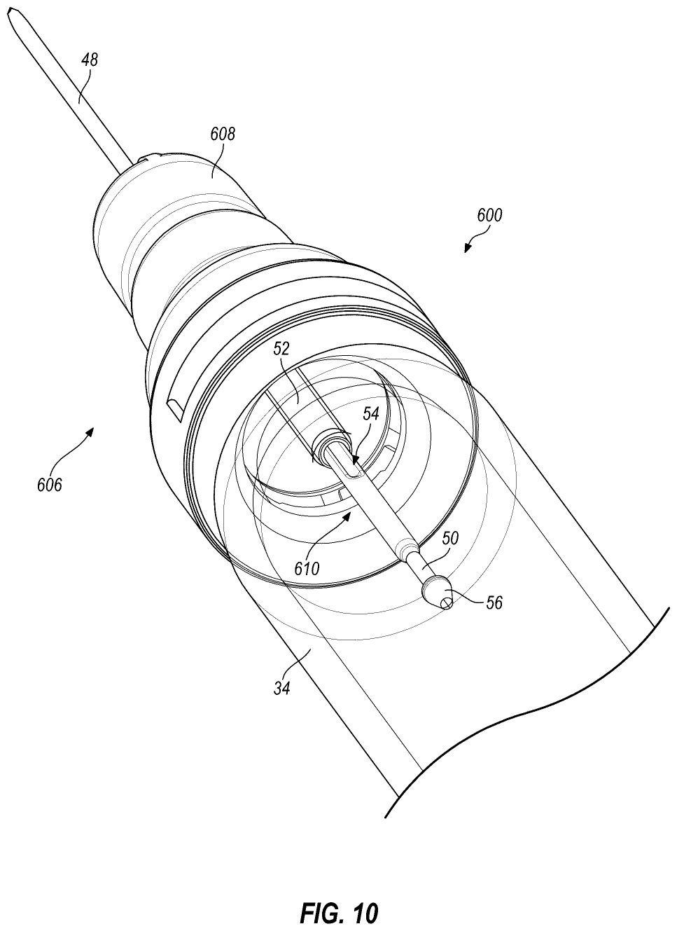

FIG. 10 is a perspective view showing the longitudinal channels (54) leading into the needle joining member (52) according to some embodiments. The needle proximal end (50) defines a radially expanded member (56) at a proximal and thereof.

While the staked needle is depicted as mounted in position, the staked needle may be removably coupled to the syringe body (34) using a Luer interface (not shown), with the proximal end (50) of the needle member extending through the Luer interface and into the syringe chamber (40). Alternatively, the needle may be fixedly or removably mounted to the flange on a cartridge body instead of a syringe. Such cartridge injection systems are disclosed in U.S. patent application Ser. No. 15/801,281, which was previously incorporated by reference herein. Alternatively, the needle may be fixedly or removably mounted to the tip of a dual chamber injection system. Such dual chamber injection systems are disclosed in U.S. patent application Ser. No. 16/435,429, which was previously incorporated by reference herein. Alternatively, the needle may be fixedly or removably mounted to the tip of a sequential injection system. Such sequential injection systems are disclosed in U.S. patent application Ser. No. 17/364,546, which was previously incorporated by reference herein. In the embodiment depicted in FIGS. 6 and 15, a significant portion of the safe needle retraction hardware resides in the plunger interior (70) defined by the plunger housing (69).

### Exemplary Dual Spring Latch Retraction Member For Use With Safe Injection Systems

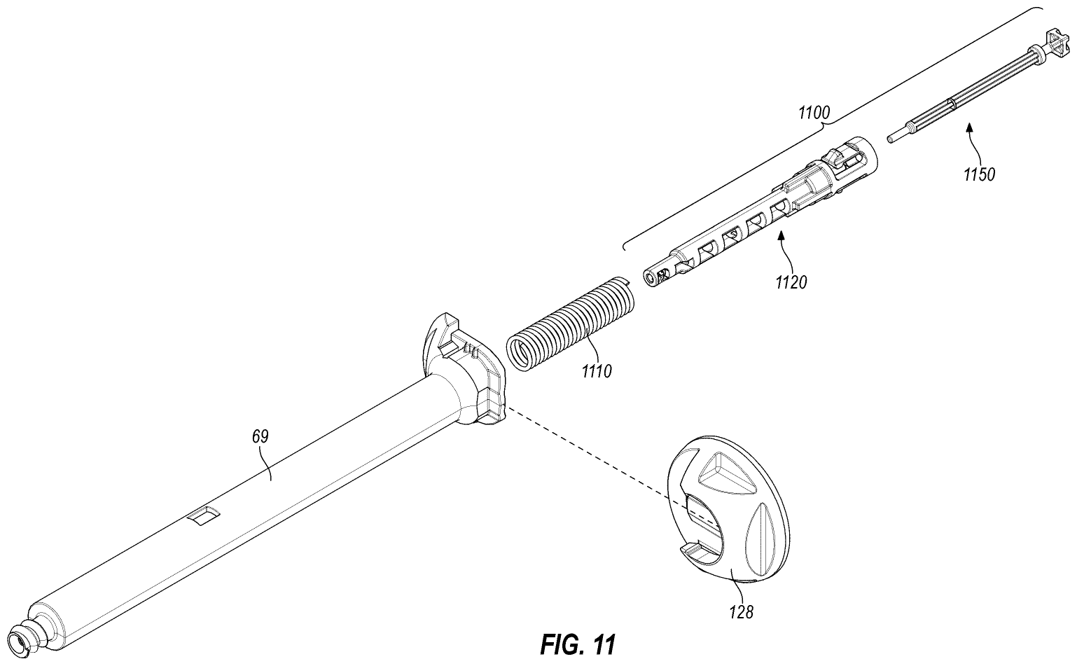

FIG. 11 is an exploded view of a retraction member (1100) for use with safe injection systems such as the system (600) depicted in FIG. 6 according to some embodiments. The retraction member (1100) includes a spring (1110) shown in the compressed state, a catch-latch (1120), and a trigger (1150). Most of the components of the retraction member (1100) are disposed in the plunger interior (70; see FIG. 15) defined by the plunger body (69). As such, the plunger member (44) has approximately the same dimensions as a conventional plunger member and is usable with the conventional syringe body (34) and conventional stopper member (36). The spring (1110) is held in a compressed state by the catch-latch (1120) and the trigger (1150), which interfere with the plunger body (69) as described below to prevent the spring (1110) from expanding proximally.

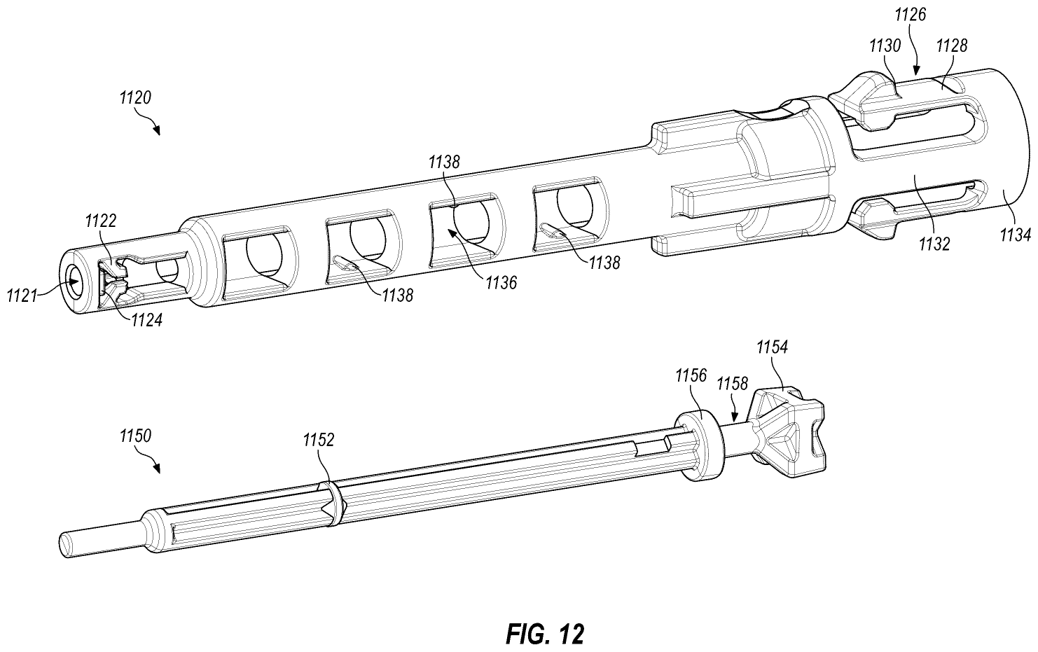

FIG. 12 is a perspective view of a catch-latch (1120) and a trigger (1150) before assembly according to some embodiments. The catch-latch (1120) is a generally tubular body that defines a pair of rotatable needle latch arms (1122) configured to interfere with the radially expanded member (56) to allow the radially expanded member (56) to move proximally rotatable needle latch arms (1122) while preventing the radially expanded member (56) from moving distally past the plurality of rotatable needle latch arms (1122). As such, the needle latch arms (1122) allow the radially expanded member (56) to be caught or captured by the catch-latch (1120). The catch-latch (1120) also includes a distal block (1124) configured to interfere with the needle latch arms (1122) to prevent rotation thereof distally past the distal block (1124). As such, the distal block (1124) prevents the needle latch arms (1122) from releasing a captured radially expanded member (56). The distal block (1124) defines an opening (1121) configured to receive the needle assembly (610). The needle latch arms (1122) and the distal block (1124) therefore cooperate to allow the catch-latch (1122) to catch or capture the radially expanded member (56) and the needle assembly (610) coupled thereto such that proximal movement of the catch-latch (1122) retracts the needle assembly (610) proximally and at least partially into the plunger interior (70).

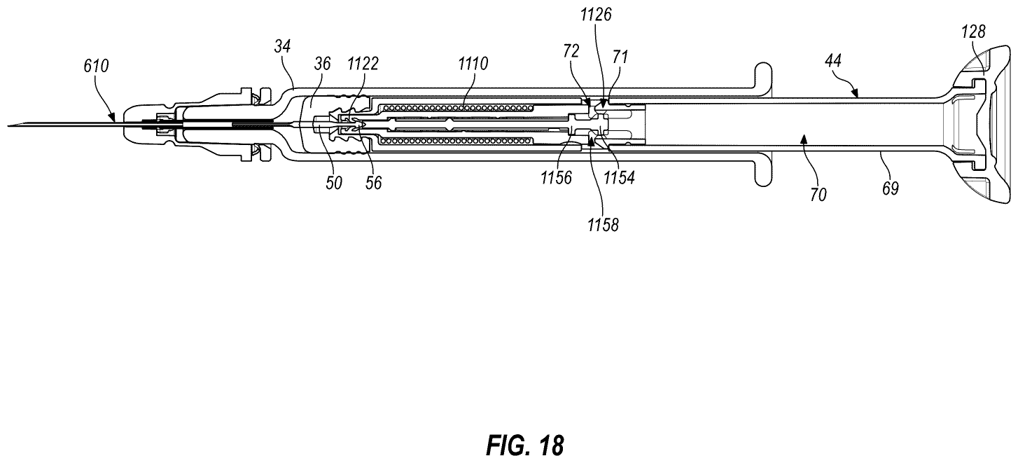

The catch-latch (1120) also defines a pair of rotatable spring latches (1126) configured to interfere with the plunger body (69) as described below to prevent the spring (1110), which is compressed by the catch-latch (1120), from expanding proximally. Each of the spring latches (1126) includes a notch (1128) at a proximal end thereof configured to facilitate rotation of the spring latch (1126) radially into an out of the catch-latch (1120). Each of the spring latches (1126) defines a proximally facing ramp (1130) configured to cooperate with each of a plurality of distally facing surfaces forming a respective plurality of openings (72) in the plunger body (69) (see FIGS. 18 to 20) to translate a proximally directed force from the spring (1110) to a radially inwardly directed force to push a distal end of the spring latch (1126) radially inward.

Having a pair of spring latches (1126) disposed on opposite sides of the catch-latch (1120) evenly distributes the force generated by the spring (1110) to the plunger member (44). Such even distribution of force prevents warping of the plunger member (44) over time while the safe injection system (600) is stored before use.

The catch-latch (1120) also defines first and second longitudinal pillars (1132) disposed on radially opposite sides of the catch-latch (1120). The catch-latch (1120) further defines a ring (1134) coupled to respective proximal ends of the first and second longitudinal pillars (1132). The ring (1134) is disposed just proximal of the spring latches (1126). The ring (1134) is configured to tilt orthogonal to the first and second longitudinal pillars (1132) to align the catch-latch (1120) and generally the retraction member (1100) with the plunger body (69). As such, the ring (1134) can compensate for unequal force applied by respective ones of the pair of spring latches (1126).

The catch-latch (1120) further defines a plurality of windows (1136) to facilitate assembly of the catch-latch (1120) and the trigger (1150). Moreover, the catch-latch (1120) defines first, second, and third radially inwardly extending bumps (1138). The trigger (1150) may include at least one radially extending ring (1152) configured to interfere with the second radially inwardly extending bumps (1138) to temporarily resist axial movement of the trigger (1150) relative to the catch-latch (1120) during assembly thereof. In an alternate embodiment, the radially extending ring (1152) may be configured to interfere with the first and/or third radially inwardly extending bumps (1138). This temporary interference places the trigger annular trough (1158) in an axial location such that the spring latches (1126) are allowed to rotate inward during assembly of the plunger rod (44). Moving the trigger (1150) distally until the distal radial extension (1156) contacts the proximally facing surface on the inner surface (1157; see FIG. 15) of the catch-latch (1120) overcomes the temporary interference and positions the trigger (1150) between the spring latches (1126), causing the spring (1100) to be held in the compressed position.

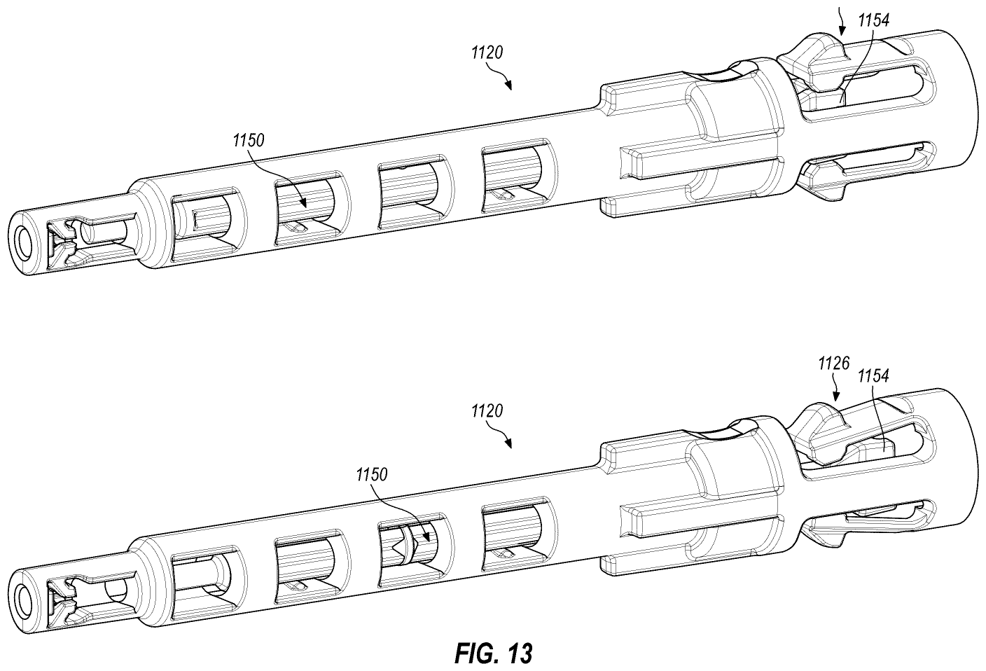

The trigger (1150) also defines proximal and distal radial extensions (1154, 1156) that define an annular trough (1158) there between. FIG. 13 is a perspective view of the catch-latch (1120) and the trigger (1150) assembled and in a latched (top figure) and unlatched (bottom figure) configuration according to some embodiments. In the latched configuration, the proximal radial extension (1154) interferes with each of the pair of spring latches (1126) to prevent rotation thereof in a radially inward direction. The catch-latch (1120) and the trigger (1150) convert from the latched configuration (top) to the unlatched configuration (bottom) with proximal movements of the trigger (1150) relative to the catch-latch (1120), which may result from pushing the plunger member (44) and the catch-latch (1120) coupled thereto distally relative to the trigger (1150). As shown in the bottom figure in FIG. 13, moving the trigger (1150) proximally relative to the catch-latch (1120) aligns the annular trough (1158) between the proximal and distal radial extensions (1154, 1156) with the spring latches (1126). This alignment allows the spring latches (1126) to rotate in a radially inward direction, effectively removing the interference between the proximally facing ramps (1130) and the distally facing surfaces forming a respective plurality of openings in the plunger body (69). Removing this interference allows the catch-latch (1120) to move proximally relative to the plunger body (69), resulting in expansion of the spring (1110) and retraction of the needle assembly (610). The distal radial extensions (1154) are shown with a slight taper, about 3 degrees per side, which when in contact with the spring latches (1126) to react against the force of the compressed spring (1100), provides a distally directed biasing force on the trigger (1150) to keep the trigger (1150) in a distal/forward position during storage, shipping, handling, and accidental dropping.

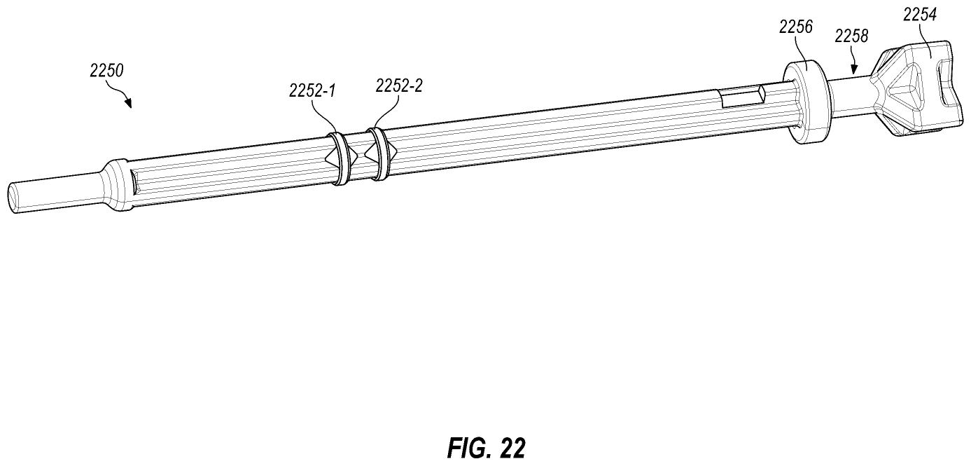

There may be two or more radially extending rings (1152) configured to interfere with the radially inwardly extending bumps (1138) to position the trigger (1150) in a longitudinal location relative to the catch-latch (1120) where the trigger (1150) is unable to slide proximally during an accidental drop of the safe injection system (600) on the thumbpad (128), thereby preventing premature release of the spring latches (1126) and the intercoupled spring (1110) (see FIGS. 22-24).

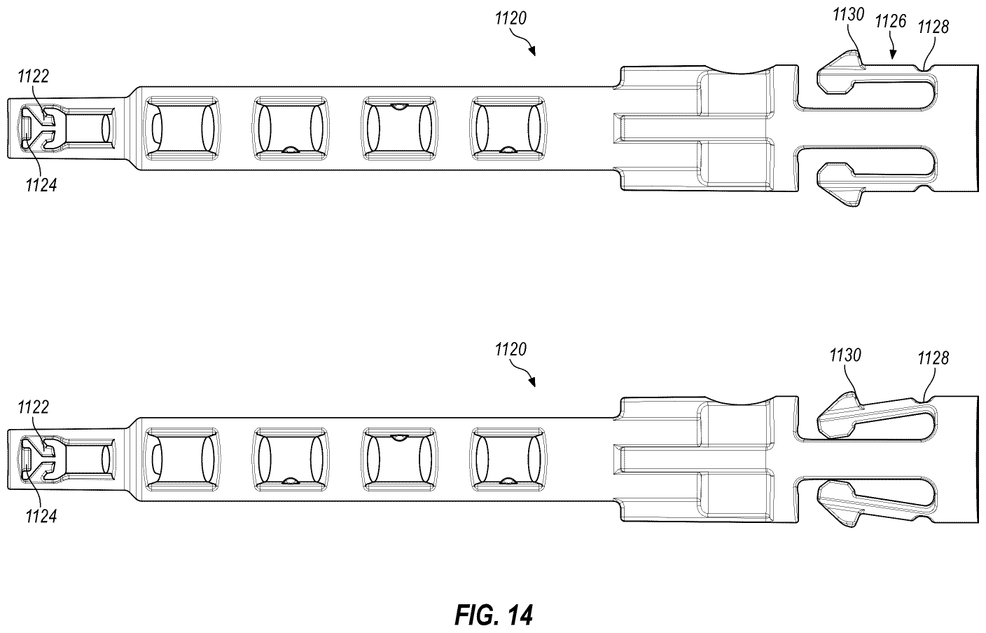

FIG. 14 is a side view of the catch-latch (1120) in a latched (top figure) and unlatched (bottom figure) configuration according to some embodiments. FIG. 14 also shows that the distal block (1124) can interfere with distal rotation of the rotatable needle latch arms (1122) to enhance grasping of the radially expanded member (56; see e.g., FIG. 10) during needle retraction.

FIGS. 15 to 21 are longitudinal cross-sectional views depicting a method of using the safe injection system (600) depicted herein to perform an injection and to retract a needle after the injection for safe disposal of the used system (600) according to some embodiments.

FIG. 15 depicts the safe injection system (600) in a transport/storage state. The syringe body (34) is prefilled with an injectable fluid (not shown). The sharp distal end of the distal needle member (48) is protected by the needle cover (63). The spring (1110) is held in a compressed state in the plunger interior (70) by interference between the spring latches (1126) and respective distally facing surfaces (71) forming respective plurality of openings (72) in the plunger body (69).

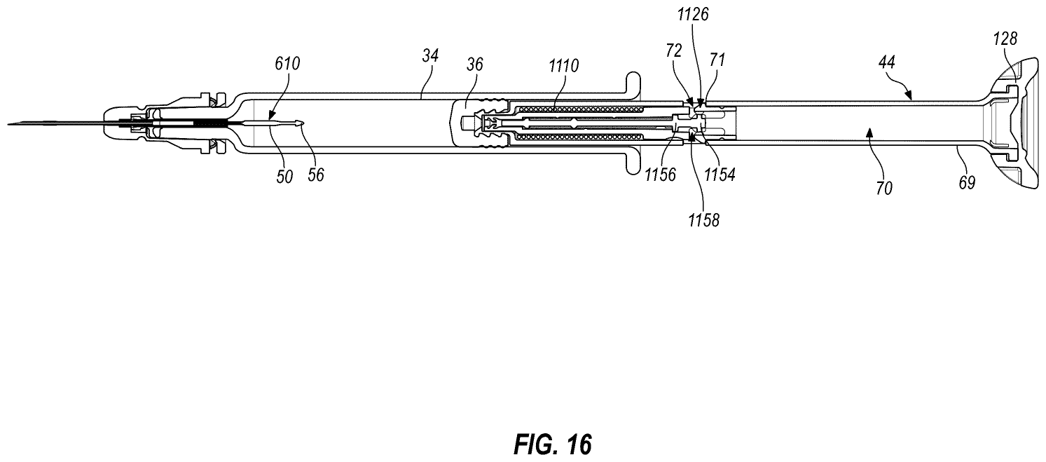

FIG. 16 depicts the safe injection system (600) in a ready to use state). The needle cover (63) has been removed from the system (600).

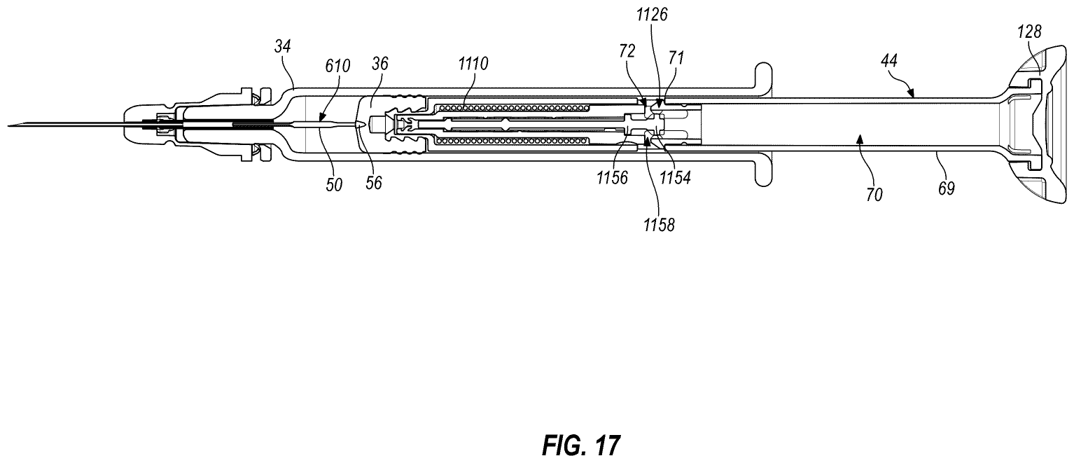

FIG. 17 depicts the safe injection system (600) after a distally directed force has been applied to the stopper member (36) through the plunger member (44), for instance by a user pressing a thumb on a plunger manipulation interface (128). The radially expanded member (56) of the needle proximal end (50) has penetrated part way into the stopper member (36) without penetrating the catch-latch (1120). The spring (1110) remains in a compressed state in the plunger interior (70).

FIG. 18 depicts the safe injection system (600) after further distally directed force has been applied to the stopper member (36) through the plunger member (44). The radially expanded member (56) of the needle proximal end (50) has moved proximally pass the needle latches (1122) in the catch-latch (1120), effectively coupling the needle assembly (610) to the catch-latch (1120). The spring (1110) remains in a compressed state in the plunger interior (70).

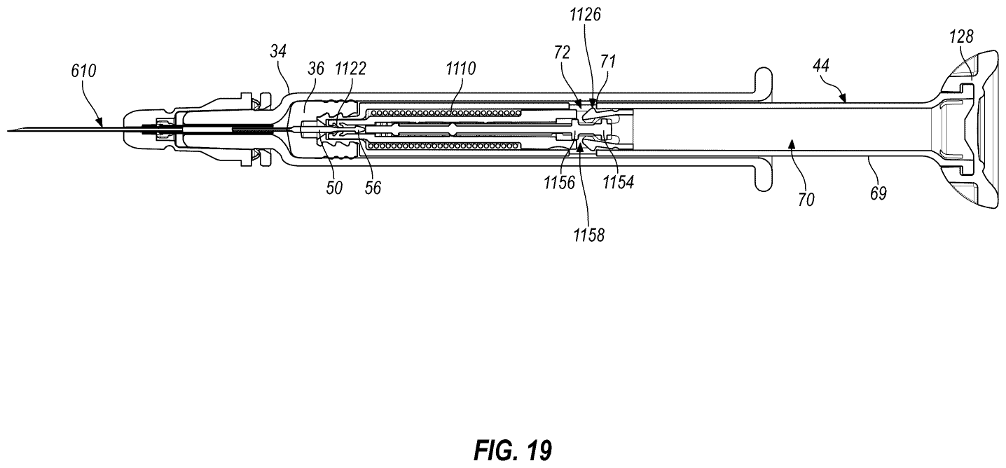

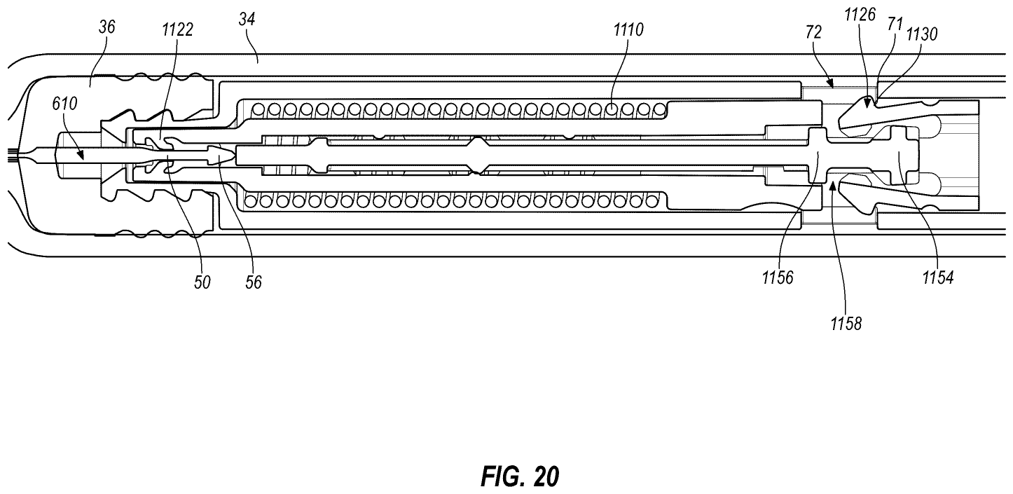

FIG. 19 depicts the safe injection system (600) after further distally directed force has been applied to the stopper member (36) through the plunger member (44), thereby moving the stopper member (36) to a distal end of the syringe body (34). Pushing the trigger (1150) against the radially expanded member (56) has moved the catch-latch (1120) distally relative to the trigger (1150), thereby aligns the annular trough (1158) between the proximal and distal radial extensions (1154, 1156) with the spring latches (1126). This alignment allows the spring latches (1126) to rotate in a radially inward direction, effectively removing the interference between the proximally facing ramps (1130) and the distally facing surfaces forming a respective plurality of openings in the plunger body (69). Removing this interference allows the catch-latch (1120) to move proximally relative to the plunger body (69), resulting in expansion of the spring (1110) and retraction of the needle assembly (610). FIG. 20 depicts the safe injection system (600) in the same state as in FIG. 19 in greater detail.

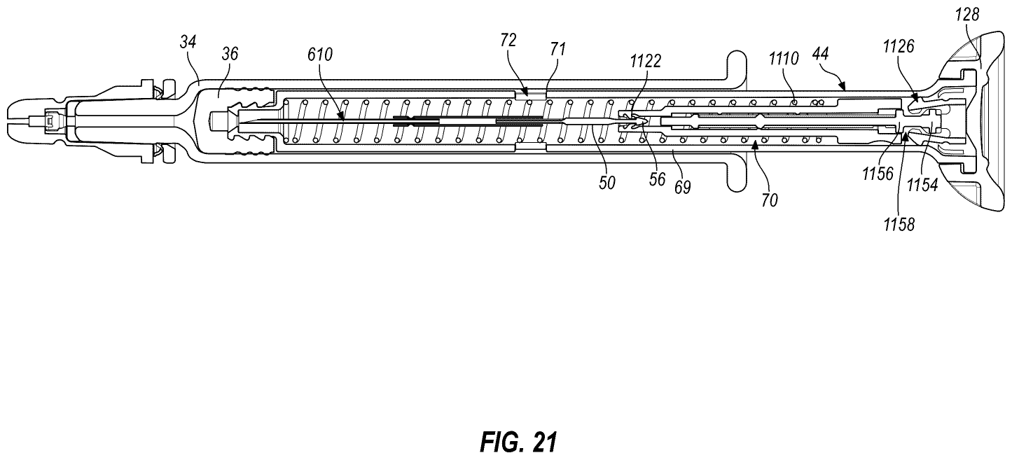

FIG. 21 depicts the safe injection system (600) after the released spring (1110) has proximally, thereby moving the catch-latch (1120) proximally in the plunger interior (70) and retracting the needle (610), including the sharp distal end thereof, into the plunger interior (70). Retracting the sharp distal end of the needle (610) into the plunger interior (70) renders the used injection system (600) safe for disposal. An alternative embodiment where the sharp distal end of the needle (610) is retracted at least into the distal tip of the syringe body luer interface (33) and/or at least into the distal tip of the needle hub (608).

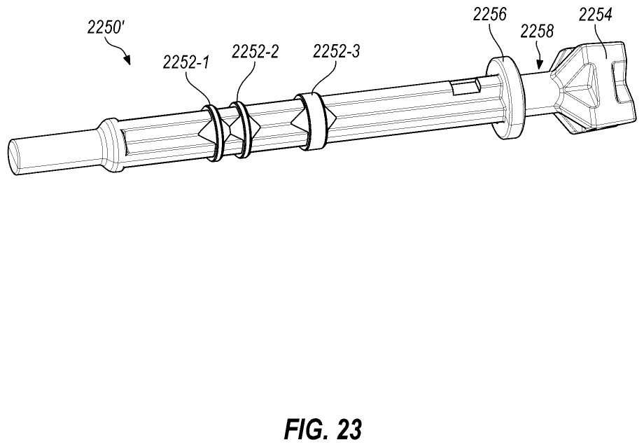

FIGS. 22 and 23 are perspective views of triggers (2250, 2250′) configured for use with spring latches (1126) such as those depicted and described herein. Similar to the trigger (1150) depicted and described herein, the triggers (2250, 2250′) define proximal and distal radial extensions (2254, 2256) that define an annular trough (2258) there between. The triggers (2250, 2250′) also include two or three radially extending rings (2252-1, 2252-2, 2252-3). The first radially extending ring (2252-1) is configured to interfere with the second radially inwardly extending bumps (1138) to temporarily resist axial movement of the triggers (2250, 2250′) relative to the catch-latch (1120) during assembly thereof. In an alternate embodiment, the first radially extending ring (2252-1) may be configured to interfere with the first and/or third radially inwardly extending bumps (1138). This temporary interference places the trigger annular trough (2258) in an axial location such that the spring latches (1126) are allowed to rotate inward during assembly of the plunger rod (44). Moving the triggers (2252, 2252′) distally until the distal radial extension (2256) contacts the proximally facing surface (1157) on the inner surface of the catch-latch overcomes the temporary interference and positions the trigger between the spring latches (2226), causing the spring (1100) to be held in the compressed position.

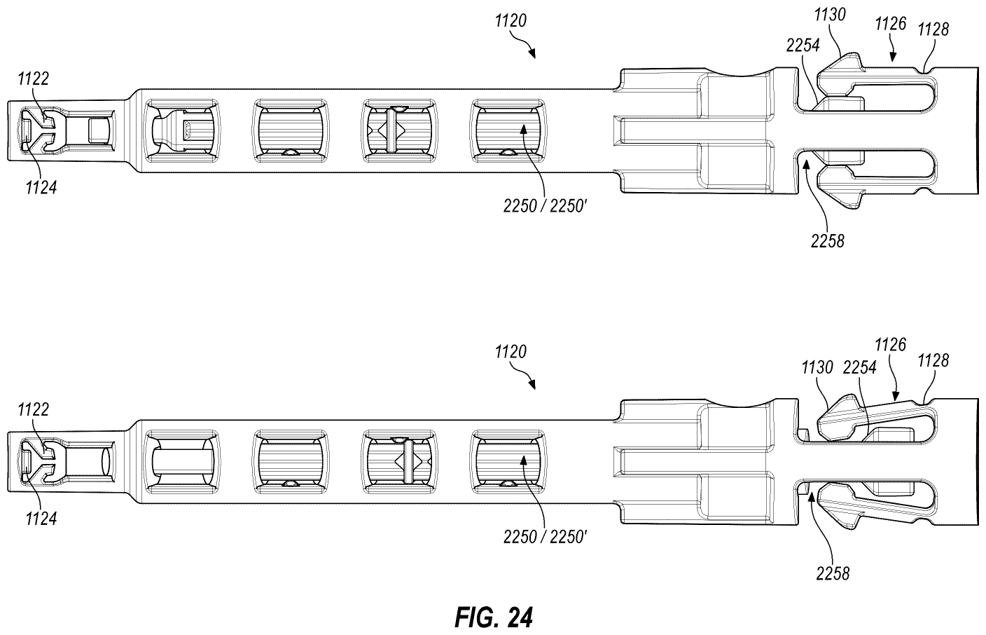

FIG. 24 is a side view of the catch-latch (1120) and one of the triggers (2250, 2250′) assembled and in a latched (top figure) and unlatched (bottom figure) configuration according to some embodiments. In the latched configuration, the proximal radial extension (2254) interferes with each of the pair of spring latches (1126) to prevent rotation thereof in a radially inward direction. The catch-latch (1120) and the triggers (2250, 2250′) convert from the latched configuration (top) to the unlatched configuration (bottom) with proximal movements of the triggers (2250, 2250′) relative to the catch-latch (1120), which may result from pushing the plunger member (44) and the catch-latch (1120) coupled thereto distally relative to the triggers (2250, 2250′). As shown in the bottom figure in FIGS. 19 and 24, moving the triggers (2250, 2250′) proximally relative to the catch-latch (1120) aligns the annular trough (2258) between the proximal and distal radial extensions (2254, 2256) with the spring latches (1126). This alignment allows the spring latches (1126) to rotate in a radially inward direction, effectively removing the interference between the proximally facing ramps (1130) and the distally facing surfaces forming a respective plurality of openings in the plunger body (69). Removing this interference allows the catch-latch (1120) to move proximally relative to the plunger body (69), resulting in expansion of the spring (1110) and retraction of the needle assembly (610). The distal radial extensions (2254) are shown with a slight taper, about 3 degrees per side, which when in contact with the spring latches (1126) to react against the force of the compressed spring (1110), provides a distally directed biasing force on the triggers (2250, 2250′) to keep the triggers (2250, 2250′) in a distal/forward position during storage, shipping, handling, and accidental dropping.

There may be two or more radially extending rings (2252-1, 2252-2, 2252-3) configured to interfere with the radially inwardly extending bumps (1138) to position the triggers (2250, 2250′) in a variety of longitudinal locations relative to the catch-latch (1120) where the triggers (2250, 2250′) is unable to slide proximally during an accidental drop of the safe injection system (600) on the thumbpad (128), thereby preventing premature release of the spring latches (1126) and the intercoupled spring (1110). The third radially extending ring (2252-3) in FIG. 23 may also be configured to perform a centering function in the interior of the catch-latch (1120). FIG. 24 also shows that the distal block (1124) can interfere with distal rotation of the rotatable needle latch arms (1122) to enhance grasping of the radially expanded member (56; see e.g., FIG. 10) during needle retraction.

While the embodiments described above include single chamber safe injection systems, the scope of the claims also include multiple chamber injection systems. For multiple chamber safety injection systems, more two or more stopper members are inserted into an injection system body (e.g., syringe body, cartridge body, etc.) to define a corresponding number of chambers.

While the injection systems depicted and described herein include syringes with staked needles, the needle cover handling devices/telescoping members described herein can be used with cartridges, an auto injector, and injection systems with Luer connectors, etc.

Various exemplary embodiments of the invention are described herein. Reference is made to these examples in a non-limiting sense. They are provided to illustrate more broadly applicable aspects of the invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. Further, as will be appreciated by those with skill in the art that each of the individual variations described and illustrated herein has discrete components and features which may be readily separated from or combined with the features of any of the other several embodiments without departing from the scope or spirit of the present inventions. All such modifications are intended to be within the scope of claims associated with this disclosure.

Any of the devices described for carrying out the subject diagnostic or interventional procedures may be provided in packaged combination for use in executing such interventions. These supply “kits” may further include instructions for use and be packaged in sterile trays or containers as commonly employed for such purposes.

The invention includes methods that may be performed using the subject devices. The methods may comprise the act of providing such a suitable device. Such provision may be performed by the end user. In other words, the “providing” act merely requires the end user obtain, access, approach, position, set-up, activate, power-up or otherwise act to provide the requisite device in the subject method. Methods recited herein may be carried out in any order of the recited events which is logically possible, as well as in the recited order of events.

Exemplary aspects of the invention, together with details regarding material selection and manufacture have been set forth above. As for other details of the present invention, these may be appreciated in connection with the above-referenced patents and publications as well as generally known or appreciated by those with skill in the art. For example, one with skill in the art will appreciate that one or more lubricious coatings (e.g., hydrophilic polymers such as polyvinylpyrrolidone-based compositions, fluoropolymers such as tetrafluoroethylene, PTFE, ETFE, hydrophilic gel or silicones) may be used in connection with various portions of the devices, such as relatively large interfacial surfaces of movably coupled parts, if desired, for example, to facilitate low friction manipulation or advancement of such objects relative to other portions of the instrumentation or nearby tissue structures. The same may hold true with respect to method-based aspects of the invention in terms of additional acts as commonly or logically employed.

In addition, though the invention has been described in reference to several examples optionally incorporating various features, the invention is not to be limited to that which is described or indicated as contemplated with respect to each variation of the invention. Various changes may be made to the invention described and equivalents (whether recited herein or not included for the sake of some brevity) may be substituted without departing from the true spirit and scope of the invention. In addition, where a range of values is provided, it is understood that every intervening value, between the upper and lower limit of that range and any other stated or intervening value in that stated range, is encompassed within the invention.

Also, it is contemplated that any optional feature of the inventive variations described may be set forth and claimed independently, or in combination with any one or more of the features described herein. Reference to a singular item, includes the possibility that there are plural of the same items present. More specifically, as used herein and in claims associated hereto, the singular forms “a,” “an,” “said,” and “the” include plural referents unless the specifically stated otherwise. In other words, use of the articles allow for “at least one” of the subject item in the description above as well as claims associated with this disclosure. It is further noted that such claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in claims associated with this disclosure shall allow for the inclusion of any additional element-irrespective of whether a given number of elements are enumerated in such claims, or the addition of a feature could be regarded as transforming the nature of an element set forth in such claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the scope of claim language associated with this disclosure.

What is claimed is:

1. A system for injection, comprising:
a syringe body having proximal and distal ends and defining a syringe interior;
a stopper member disposed in the syringe interior;
a needle hub assembly coupled to the syringe body at the distal end thereof and comprising
a needle hub, and
a needle coupled to the needle hub and having a needle proximal end;
a plunger member coupled to the stopper member and configured to be manipulated to insert the stopper member distally in the syringe interior relative to the syringe body and the needle hub,
wherein the plunger member comprises
a plunger body defining a plunger interior,
a retraction member disposed in the plunger interior and configured to couple to the needle proximal end, and
a spring disposed in the plunger interior and configured to withdraw the retraction member proximally in the plunger interior, the spring having a compressed state and a released state, and
wherein the retraction member comprises
a catch-latch having
a plurality of distal arms configured to interfere with the needle proximal end, and
a plurality of spring latches configured to hold the spring in the compressed state, and
a trigger movably disposed in the catch-latch along a longitudinal axis thereof and defining
a proximal radial extension,
a distal radial extension, and
an annular trough between the proximal and distal radial extensions.

2. The system of claim 1, wherein the plurality of spring latches comprises first and second spring latches disposed on radially opposite sides of the catch-latch.

3. The system of claim 2, wherein the first and second spring latches equally distribute a force from the spring, through the catch-latch, to the plunger body.

4. The system of claim 1, wherein the plunger body defines a plurality of openings corresponding to the plurality of spring latches and a plurality of distally facing surfaces defining respective proximal ends of respective ones of the plurality of openings,
wherein each spring latch of the plurality of spring latches comprises a proximally facing ramp configured to cooperate with a corresponding distally facing surface of a corresponding opening under a proximally directed force from the spring on the catch-latch to push the spring latch radially inward.

5. The system of claim 4, wherein each spring latch of the plurality of spring latches comprises a notch configured to facilitate rotation of the spring latch toward and away from the trigger.

6. The system of claim 1, wherein the catch-latch comprises:
first and second longitudinal pillars disposed on radially opposite sides of the catch-latch; and
a ring coupled to respective proximal ends of the first and second longitudinal pillars and disposed proximal of the plurality of spring latches.

7. The system of claim 6, wherein the ring is configured to tilt orthogonal to the first and second pillars to align the retraction member with the plunger body.

8. The system of claim 1, wherein the catch-latch defines a plurality of windows therein.

9. The system of claim 1, wherein the trigger comprises a radially extending ring, and
wherein the catch-latch comprises a plurality of radially inwardly extending bumps configured to interfere with the radially extending ring to temporarily resist axial movement of the trigger relative to the catch-latch.

10. The system of claim 9, wherein the plurality of radially inwardly extending bumps comprises first, second, and third radially inwardly extending bumps, and
wherein the second radially inwardly extending bump is disposed on an opposite side of the catch-latch from the first and third radially inwardly extending bumps.

11. The system of claim 9, wherein the radially extending ring and the plurality of radially inwardly extending bumps are configured to provide a plurality of rest configurations during assembly of the retraction member.

12. The system of claim 9, wherein the trigger comprises a second radially extending ring.

13. The system of claim 12, wherein the second radially extending ring and the plurality of radially inwardly extending bumps are configured to prevent premature conversion of the spring from the compressed state to the released state during an accidental drop of the system.

14. The system of claim 1, wherein the needle proximal end defines a radially expanded member at a proximal end thereof, and
wherein the catch-latch defines a plurality of rotatable needle latch arms configured to interfere with the radially expanded member to allow the radially expanded member to move proximally past the plurality of rotatable needle latch arms while preventing the radially expanded member from moving distally past the plurality of rotatable needle latch arms.

15. The system of claim 14, wherein the catch-latch also defines a distal block configured to interfere with the plurality of rotatable needle latch arms to prevent the plurality of rotatable needle latch arms from rotating distally past the distal block.

16. The system of claim 15, wherein the distal block defines an opening therethrough configured to receive the needle.

17. The system of claim 1, wherein the needle also has a tubular joining member configured to receive a distal end of the needle proximal end, and
wherein the needle proximal end defines one or more channels at the distal end thereof.
